(19) United States
(12) Patent Application Publication
(10) Pub. No.: US 2019/0276820 A1
(43) Pub. Date: Sep. 12, 2019

(54) SINGLE EXTRACELLULAR VESICLE MULTIPLEXED PROTEIN AND RNA ANALYSIS

(71) Applicant: Mantra Bio, Inc., San Francisco, CA (US)

(72) Inventors: Terry A. Gaige, Alameda, CA (US); Chi-Chih (Ginny) Kang, Berkeley, CA (US); Catherine RoseMary Planey, San Francisco, CA (US); Alexander Fen Mok, San Francisco, CA (US)

(73) Assignee: Mantra Bio, Inc., San Francisco, CA (US)

(21) Appl. No.: 16/351,493

(22) Filed: Mar. 12, 2019

Related U.S. Application Data
(60) Provisional application No. 62/641,796, filed on Mar. 12, 2018.

(51) Int. Cl. C12N 15/10

(52) U.S. Cl. CPC C12N 15/1065; C12N 15/1096

(57) ABSTRACT

A system for identifying exosome subpopulations and payloads for potential targeted therapeutics comprises a simultaneous extracellular vesicle membrane and enclosed content detecting composition. The extracellular vesicle membrane and enclosed content detecting composition includes: a bead comprising a selective binding agent; an extracellular vesicle membrane protein-specific DNA tag bound to the selective binding agent and comprising a poly-T end; and an extracellular vesicle-derived RNA comprising a poly-A end bound to the membrane protein-specific DNA tag poly-T end. The composition may comprise cDNA bound to the RNA. The selective binding agent may comprise a barcode and a molecular identifier. The selective binding agent may selectively bind the membrane protein identifier DNA present in a droplet. In some embodiments, the bead may be a magnetic bead. In some embodiments, the bead may be a conductive bead.

### CROSS-REFERENCE TO RELATED APPLICATIONS

This application claims the benefit of U.S. Provisional Application No. 62/641,796 (Attorney docket MBIOP002P), entitled “SINGLE EXTRACELLULAR VESICLE MULTIPLEXED PROTEIN AND RNA ANALYSIS,” filed on Mar. 12, 2018, which is incorporated by reference herein in its entirety for all purposes

### TECHNICAL FIELD

The present disclosure relates generally to multivesicular endosomes and exosomes, and more specifically to identifying exosome subpopulations and payloads for potential targeted therapeutics.

### BACKGROUND

Extracellular vesicles (EVs) vary widely in their characteristics. A specific subset of EVs, termed exosomes and characterized by the specific cell pathway in which they are emitted, are generally expected to be in the size range of 40-100 nm and are known to carry a mixture of protein, RNA and genomic DNA. The function of exosomes is not yet clearly known but they have been demonstrated to participate in cell-to-cell signaling as they are transferred between cells and influence the behavior of the receiving cell.

Exosomes are released from the cells when a multivesicular endosomes (MVE) fuse with the cytoplasmic membrane to release their vesicle content from the cells instead of merging with a lysosome for degradation. Various subpopulations of exosomes target tissues differently. Exosome subpopulations are differentiated by one of many properties; such as their cell of origin, their surface proteins, or their size. Because of the small size, sheer number and great heterogeneity of exosomes they have been difficult to characterize.

Therefore, there is a need for better and more efficient systems and methods for identifying exosome subpopulations and payloads for potential targeted therapeutics.

### SUMMARY

The following presents a simplified summary of the disclosure in order to provide a basic understanding of certain embodiments of the disclosure. This summary is not an extensive overview of the disclosure and it does not identify key/critical elements of the disclosure or delineate the scope of the disclosure. Its sole purpose is to present some concepts disclosed herein in a simplified form as a prelude to the more detailed description that is presented later.

In general, certain embodiments of the present disclosure provides systems and methods for identifying exosome subpopulations and payloads. In one aspect, which may include at least a portion of the subject matter of any of the preceding and/or following examples and aspects, a simultaneous extracellular vesicle membrane and enclosed content detecting composition comprises: a bead comprising a selective binding agent; an extracellular vesicle membrane protein-specific DNA tag bound to the selective binding agent and comprising a poly-T end; and an extracellular vesicle-derived RNA comprising a poly-A end bound to the membrane protein-specific DNA tag poly-T end.

The composition may comprise cDNA bound to the RNA. The selective binding agent may comprise a barcode and a molecular identifier. The selective binding agent may selectively bind the membrane protein identifier DNA present in a droplet. In some embodiments, the bead may be a magnetic bead. In some embodiments, the bead may be a conductive bead.

Further provided is a method of simultaneously detecting extracellular vesicle membrane and enclosed content, as described herein. The method comprises binding tagging complexes each comprising a membrane protein-specific DNA tag bound to a membrane protein-selective antibody to a population of extracellular vesicles; encapsulating a bead and one of the extracellular vesicles comprising a tagging complex in a droplet; removing the tagging complex from the extracellular vesicle and the DNA tag from the antibody; binding the DNA tag to the bead; lysing the extracellular vesicle; binding RNA derived from inside the extracellular vesicle to the DNA tag bound to the bead to form a detection complex; and simultaneously amplifying the RNA and the DNA tag of the detection complex and thereby simultaneously detecting extracellular vesicle membrane content and enclosed content.

The method may further comprise isolating the population of extracellular vesicles. A portion of extracellular vesicles of the population of extracellular vesicles may be free of encapsulated RNA.

The amplification may only be performed on samples in droplets comprising an RNA derived from inside the extracellular vesicle and a DNA tag. In some embodiments, the amplification may preferentially performed on samples in droplets comprising an RNA derived from inside the extracellular vesicle and DNA tag.

The DNA tag may comprise a poly-T end and the RNA may comprise a poly-A end. Thus, binding the RNA to the DNA tag comprises binding the poly-T end of the DNA tag with the poly-A end of the RNA.

The method may further comprise isolating a droplet comprising a bead and one of the extracellular vesicles comprising a tagging complex in a well of a droplet isolating device. The bead may be a magnetic bead and the droplet isolating device comprises a magnet that positions the beads inside the well.

The method may further comprise purifying RNA derived from inside the extracellular vesicle after the extracellular vesicle is lysed. Lysing the extracellular vesicle may produce a lysed solution and wherein purifying the RNA comprises treating the lysed solution with proteinase K.

The method may further comprise synthesizing a cDNA on the RNA. The method may further comprise cleaving the detection complex from the bead before simultaneously amplifying the RNA and the DNA tag.

In some embodiments, the extracellular vesicles may be exosomes. In some embodiments, the extracellular vesicles may be nanovesicles. The droplet may be immiscible with a surrounding carrier fluid. The droplet may comprise an aqueous fluid.

The bead may comprise a selective binding agent and binding the DNA tag to the bead comprises binding the DNA tag to the selective binding agent. The selective binding agent may comprise a barcode bound to a molecular identifier.

In another described aspect, a method of detecting extracellular vesicle content comprises: binding tagging complexes each comprising a membrane protein-specific DNA tag bound to a membrane protein-selective antibody to a population of extracellular vesicles, wherein a first portion of the population are extracellular vesicles comprising a nucleic acid payload and wherein a second portion of the population are extracellular vesicles free of a nucleic acid payload; encapsulating a bead and one of the extracellular vesicles comprising a tagging complex in a droplet; removing the tagging complex from the extracellular vesicle and the DNA tag from the antibody; binding the DNA tag to the bead; lysing the extracellular vesicle; and binding RNA derived from inside the extracellular vesicle to the DNA tag bound to the bead to form a detection complex, and simultaneously amplifying the RNA and the DNA tags of the detection complex if the extracellular vesicle is from the first portion; or discarding the RNA and the DNA tags if the extracellular vesicle is from the second portion.

These and other embodiments are described further below with reference to the figures.

### BRIEF DESCRIPTION OF THE DRAWINGS

The disclosure may best be understood by reference to the following description taken in conjunction with the accompanying drawings, which illustrate particular embodiments of the present disclosure.

FIG. 1 illustrates a flow process of a first implementation for identification of EV subpopulations, in accordance with one or more embodiments.

FIG. 2 illustrates a flow process of a second implementation for identification of EV subpopulations, in accordance with one or more embodiments.

FIG. 3 illustrates a flow process of a third implementation for identification of EV subpopulations, in accordance with one or more embodiments.

FIG. 4 illustrates the various configurations of a barcoded bead with DNA-tags, in accordance with one or more embodiments.

FIGS. 5A-5E illustrate a method for single-cell protein profiling, in accordance with one or more embodiments.

FIG. 6 illustrates a method for single-molecule droplet barcoding (SMDB), in accordance with one or more embodiments.

FIG. 7 illustrates a method for simultaneously detecting extracellular vesicle membrane and enclosed content, in accordance with one or more embodiments.

FIG. 8 illustrates another method for detecting extracellular vesicle content, in accordance with one or more embodiments.

### DESCRIPTION OF PARTICULAR EMBODIMENTS

Reference will now be made in detail to some specific examples of the present disclosure including the best modes contemplated by the inventors for carrying out the present disclosure. Examples of these specific embodiments are illustrated in the accompanying drawings. While the present disclosure is described in conjunction with these specific embodiments, it will be understood that it is not intended to limit the present disclosure to the described embodiments. On the contrary, it is intended to cover alternatives, modifications, and equivalents as may be included within the spirit and scope of the present disclosure as defined by the appended claims.

In the following description, numerous specific details are set forth in order to provide a thorough understanding of the present disclosure. Particular example embodiments of the present disclosure may be implemented without some or all of these specific details. In other instances, well known process operations have not been described in detail in order not to unnecessarily obscure the present disclosure.

Various techniques and mechanisms of the present disclosure will sometimes be described in singular form for clarity. However, it should be noted that some embodiments include multiple iterations of a technique or multiple instantiations of a mechanism unless noted otherwise. Furthermore, the techniques and mechanisms of the present disclosure will sometimes describe a connection between two entities. It should be noted that a connection between two entities does not necessarily mean a direct, unimpeded connection, as a variety of other entities may reside between the two entities. Consequently, a connection does not necessarily mean a direct, unimpeded connection unless otherwise noted.

The general purpose of the present disclosure, which will be described subsequently in greater detail, is to provide an improved system and method for identifying exosome subpopulations and payloads. Exosomes are a specific subset of EVs that are characterized by the specific cell pathway in which they are emitted. As used herein the term “extracellular vesicles” (“EV”) may be used interchangeably with the term “exosomes.” As used herein, the term “vesicle” can also be used to refer to a “single cell.” In other words, processes that involve vesicles can also be applied to single cells.

Extracellular vesicles (EVs) are a class of membrane bound organelles secreted by various cell types. By “extracellular vesicle” as provided herein is meant a cell-derived vesicle having a membrane that surrounds and encloses a central internal space. Membranes of EVs can be composed of a lipid bi-layer having an external surface and an internal surface bounding an enclosed volume. As described further below, such membranes can have one or more types of cargo, such as proteins, embedded therein. EVs include all membrane-bound vesicles that have a cross-sectional diameter smaller than the cell from which they are secreted. EVs can have a longest dimension, such as a longest cross-sectional dimension, such as a cross-sectional diameter ranging from 10 nm to 1000 nm, such as 20 nm to 1000 nm, such as 30 nm to 1000 nm, such as 10 to 100 nm, such as 20 to 100 nm, such as 30 to 100 nm, such as 40 to 100 nm, such as 10 to 200 nm, such as 20 to 200 nm, such as 30 to 200 nm, such as 40 to 200 nm, such as 10 to 120 nm, such as 20 to 120 nm, such as 30 to 120 nm, such as 40 to 120 nm, such as 10 to 300 nm, such as 20 to 300 nm, such as 30 to 300 nm, such as 40 to 300 nm, such as 50 to 1000 nm, such as 500 to 2000 nm, such as 100 to 500 nm, such as 500 to 1000 nm and such as 40 nm to 500 nm, each range inclusive.

The term “membrane” as used in the subject disclosure, refers to a boundary layer separating an interior vesicle space from an exterior space, wherein the layer includes one or more biological molecules such as lipids, and in some instances, carbohydrates and/or polypeptides. Membranes can include lipids and/or fatty acids. Such lipids can include phospholipids, phosphatidylserine, sphingolipids, sterols, glycolipids, fatty acids, cholesterols, and/or phosphoglycerides. Membranes can also include one or more polypeptide and/or polysaccharide, e.g., glycan.

EVs include (i) extravesicles: 30-150 nanometer diameter membraneous vesicles of endocytic origin (ii) ectosomes (also referred to as shedding microvesicles, SMVs): large membranous vesicles (ranging, for example, from 50 nm to 5000 nm in diameter) that are shed directly from the cellular plasma membrane and (iii) apoptotic blebs (ranging, for example, from 50 nm to 5000 nm in diameter): released by dying cells.

EVs, particularly extravesicles, are important for intercellular communications within the human body and involved in many pathophysiological conditions such as Cancer or neurodegenerative disease. EVs are abundant in various patient biological samples, e.g., biological fluids, including but not limited to blood, urine, saliva, cerebrospinal fluid, breast milk, synovial, amniotic, and lymph fluids.

In various aspects, EVs include cell fragments. EVs are derived from, such as by being produced and released by producer donor cells. The term “producer cell,” as used herein, refers to a cell from which an EV can be extracted or isolated. Producer cells are cells which act as a source for one or more EVs. Producer cells can share one or more component, such as a nucleic acid molecule, lipid, protein, lipid, and/or sugar component with derivative EVs. Producer cells can also be isolated and/or cultured cells. Producer cells can, in some aspects be modified or synthetic cells. Producer cells can be immune cells. In various instances a producer cell is a primary cell or a cell line.

As used in the subject disclosure, the terms “extracted,” “extracting,” “isolate,” “isolated,” “isolating,” “purify,” “purified,” and “purifying,” refer to a stage of a preparation of desired subject EVs, that have been subjected to one or more purification process, such as an enrichment and/or selection of the desired EV preparation. Also, a preparation of EVs can be a plurality of unknown or known amount and/or concentration. In various instances, purifying or isolating is the process of removing, such as partially removing or substantially removing, a portion (e.g. a fraction) of the EVs from a sample containing one or more biological components, such as producer cells. In various aspects, an EV composition that has been isolated is enriched as compared to the starting fraction, e.g., producer cell preparations), from which the EV composition is obtained. Such enrichment can, for example, be enrichment by 5%, 10%, 20%, 30%, 40%, 50%, 60%, 70%, 80%, 90%, 95%, 96%, 97%, 98%, 98.5%, 99%, 99.5%, 99.9%, 99.99%, 99.999%, 99.9999%, or 99.9999% or greater, as compared to the starting fraction. In some instances, an isolated EV sample has an amount and/or concentration of desired EVs at or above an acceptable concentration and/or amount. According to some versions, isolated EV preparations are free or substantially free of residual biological products. In some aspects, isolated EV preparations are 100% free, 99.5% free, 99% free, 98.5% free, 98% free, 97% free, 96% free, or 95% free, or 90% or greater free, of any contaminating biological matter such as producer cells. Undesired residual biological aspects can include unwanted nucleic acids, proteins, lipids, and/or or metabolites or abiotic materials such as including chemicals. The phrase substantially free of residual biological products can also mean that the EV composition contains no producer cells that are detectable and that only EVs in the composition are detectable. An isolated EV composition in various aspects, has no undesired activity that is detectable or, the level or amount of the detected undesired activity is at or below an acceptable level.

Also, the phrases “nucleic acid molecule,” and “nucleic acid” as used herein refer to a double or single-stranded polymer of ribonucleotide or deoxyribonucleotide bases. A nucleic acid can be recombinant and peptides, e.g., exogenous polypeptides, can be expressed when the nucleic acid is introduced into a cell. Nucleic acids can, for example, include vectors, messenger RNA (mRNA), single stranded RNA that is complementary to an mRNA (antisense RNA), microRNA (mi RNA), tRNA, small interfering RNA (siRNA), small or short hairpin RNA (shRNA), long non-coding RNA (lncRNA), chromosomal DNA, e.g., double stranded DNA (dsDNA), and/or self-replicating plasmids.

EVs can also be derived from cells by manipulation, such as indirect or direct manipulation, e.g., by extrusion or application of alkaline solutions. EVs can include organelles separated into vesicles, and vesicles produced by living cells such as by fusion of a late endosome with the plasma membrane or direct plasma membrane budding. Furthermore, EVs can be derived from a dead or living organism, cultured cells, explanted tissues or organs, or any combination thereof.

In various aspects, EVs include a cargo including, for example, a receiver, or a targeting moiety for binding a target. A “receiver,” as used herein, refers to a molecule that promotes the interaction, e.g., binding, of an EV with a target, and/or directs an EV to a target. A receiver can be a polypeptide and/or an antibody. As used herein, a “target” is a cell, a pathogen, a metabolite, a polypeptide complex, or any molecule or structure that resides in a tissue or circulates in the circulatory system or lymphatic system of the subject, such as an immune cell or a cancer cell. A target can be any of such aspects which readily interacts with, e.g., binds, a receiver.

EVs can also include a payload, e.g. a therapeutic agent, a sugar, e.g. a glycan, simple sugar, polysaccharide, a polynucleotide, e.g. a nucleic acid, DNA and/or RNA, other molecules, or any combination thereof. The term “payload” as applied herein refers to an agent, e.g., a therapeutic agent, that acts on a target, such as a cell, that is contacted with and/or bound to an EV. Further examples of payloads include amino acids such as amino acids having a detectable moiety or a toxin or that disrupt translation, polypeptides such as enzymes, nucleotides having a detectable moiety or a toxin or that disrupt transcription, nucleic acids that encode a polypeptide such as an enzyme, or RNA molecules that have regulatory function such as dsDNA, miRNA, siRNA, and lncRNA, small molecules such as small molecule toxins and drugs, lipids, and/or carbohydrates.

Also, as referred to in the subject disclosure, “therapeutic molecules,” or “therapeutic agents,” which are also referred to as “therapeutics,” are molecules or compounds that when present in an effective amount, produce a desired therapeutic effect on a subject in need thereof. Such an effect can be physiologic and/or pharmacologic. Therapeutics include one or more compounds, for example, a small molecule drug, or a biologic, such as a polypeptide drug or a nucleic acid drug, that when administered to a subject has a conveyable and/or measurable effect on the subject. Such an effect can be that it treats, such as decreases or alleviates, one or more symptom of a condition, disease, or disorder.

EVs as provided herein include exosomes. By “exosome” is meant a cell-derived vesicle composed of a membrane enclosing an internal space, wherein the vesicle is generated from a cell by fusion of the late endosome with the plasma membrane or by direct plasma membrane budding, and wherein the vesicle has a longest dimension, such as a longest cross-sectional dimension, such as a cross-sectional diameter, ranging for example, from 10 nm to 150 nm, such as 20 nm to 150 nm, such as 20 nm to 130 nm, such as 20 nm to 120 nm, such as 20 to 100 nm, such as 40 to 130 nm, such as 30 to 150 nm, such as 40 to 150 nm, or from 30 nm to 200 nm, such as 30 to 100 nm, such as 30 nm to 150 nm, such as 40 nm to 120 nm, such as 40 to 150 nm, such as 40 to 200 nm, such as 50 to 150 nm, such as 50 to 200 nm, such as 50 to 100 nm, or from 10 to 400 nm, such as 10 to 250 nm, such as 50 to 250 nm, such as 100 to 250 nm, such as 200 to 250 nm, such as 10 to 300 nm, such as 50 to 400 nm, such as 100 to 400 nm, such as 200 to 400 nm, each range inclusive. As used herein, “inclusive” refers to a provided range including each of the listed numbers. Unless noted otherwise herein, all provided ranges are inclusive.

Exosomes can be derived from a producer cell, and/or isolated from the producer cell based on one or more exosome isolating characteristics, such as density, size, biochemical parameters, or any combination thereof. In various embodiments, exosome generation does not destroy the exosome-producing cell. Exosomes can include lipids or fatty acids and polypeptides. In various aspects, exosomes include a cargo including, for example, a receiver, e.g. a targeting moiety, a payload, e.g. a therapeutic agent, a sugar, e.g. a glycan, simple sugar, polysaccharide, a polynucleotide, e.g. a nucleic acid, DNA and/or RNA, and/or other molecules, or any combination thereof. In some embodiments, EVs such as exosomes are free of and do not include genetic material such as nucleic acids therein.

EVs as provided herein include nanovesicles. By “nanovesicle” is meant a cell-derived vesicle composed of a membrane enclosing an internal space, wherein the vesicle is generated from a cell by manipulation, e.g., indirect or direct manipulation, such that the vesicle would not be produced by the cell without the manipulation, and wherein the vesicle has a longest dimension, such as a longest cross-sectional dimension, such as a cross-sectional diameter, ranging for example, from 10 nm to 300 nm, such as such 20 nm to 300 nm, such as 20 nm to 275 nm, such as 20 nm to 250 nm, such as 20 nm to 200 nm, such as 30 nm to 175 nm, such as 30 nm to 150 nm, such as 30 nm to 120 nm, such as 30 nm to 110 nm, each range inclusive. Cell manipulation for nanovesicle production can include application of alkaline solutions, serial extrusion, sonication, or any combinations thereof. In various aspects, production of a nanovesicle results in destruction of the producer cell. Nanovesicles can be derived from a producer cell, and/or isolated from the producer cell based on one or more nanovesicle isolating characteristics, such as density, size, biochemical parameters, or any combination thereof. In some aspects, concentrations of nanovesicles are free or substantially free of EVs that are derived from producer cells by fusion of a late endosome with the plasma membrane or by budding directly from the plasma membrane. Nanovesicles can include lipids or fatty acids and polypeptides. In various aspects, nanovesicles include a cargo including, for example, receiver, e.g. a targeting moiety, a payload, e.g. a therapeutic agent, a sugar, e.g. a glycan, simple sugar, polysaccharide, a polynucleotide, e.g. a nucleic acid, DNA and/or RNA, and/or other molecules, or any combination thereof.

Simultaneous multiplexed measurement of surface proteins and nucleic acids on single extracellular vesicles (EV). Unique DNA tags corresponding to different surface proteins and EV RNA fragments (consisting of any combination or single instance of type of RNA, such as mRNAs or microRNAs) and/or EV DNA fragments will be analyzed by next-generation sequencing. The described systems and methods may be applied to both human bio-fluid samples and cell culture samples.

Extracellular vesicles (EVs) vary widely in their characteristics. A specific subset of EVs, termed exosomes and characterized by the specific cell pathway in which they are emitted, are generally expected to be in the size range of 40-100 nm and are known to carry a mixture of protein, RNA and genomic DNA. The function of exosomes is not yet clearly known but they have been demonstrated to participate in cell-to-cell signaling as they are transferred between cells and influence the behavior of the receiving cell.

Exosomes are released from the cells when a multivesicular endosomes (MVE) fuse with the cytoplasmic membrane to release their vesicle content from the cells instead of merging with a lysosome for degradation.

Researchers have shown that various subpopulations of exosomes target tissues differently. Exosome subpopulations are differentiated by one of many properties; such as their cell of origin, their surface proteins, or their size. The ability for exosomes to selectively target tissues is likely largely mediated by the accessible surface proteins which a receiving cell can interact with to either accept, ingest and process; or reject and return to circulation. Because of the small size, sheer number and great heterogeneity of exosomes they have been difficult to characterize. The described systems and methods address this issue with increased sensitivity and multiplexing capability, thereby enabling the identification of exosome subpopulations and payloads for potential targeted therapeutics and/or more precise biomarkers for clinical applications.

Other objects and advantages of the present apparatus, systems, and methods will become obvious to the reader and it is intended that these objects and advantages are within the scope of the present disclosure.

To the accomplishment of the above and related objects, the disclosed apparatus, systems and methods may be embodied in the form illustrated in the accompanying drawings, attention being called to the fact, however, that the drawings are illustrative only, and that changes may be made in the specific construction illustrated.

Turning now descriptively to the drawings, in which similar reference characters denote similar elements throughout the several views, the attached figures illustrate systems and methods for identifying EV subpopulations, including exosome subpopulations, and payloads. Several iterations or possible implementations of the technology are provided in the following figures. The figures assume that the EVs have already been isolated. The addition of EV isolation to the disclosed single droplet workflow is a novel embodiment.

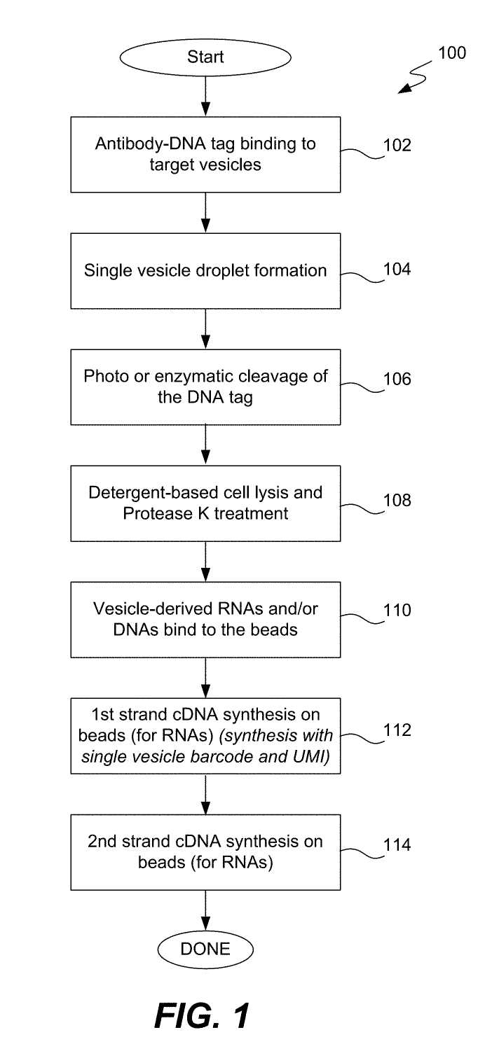

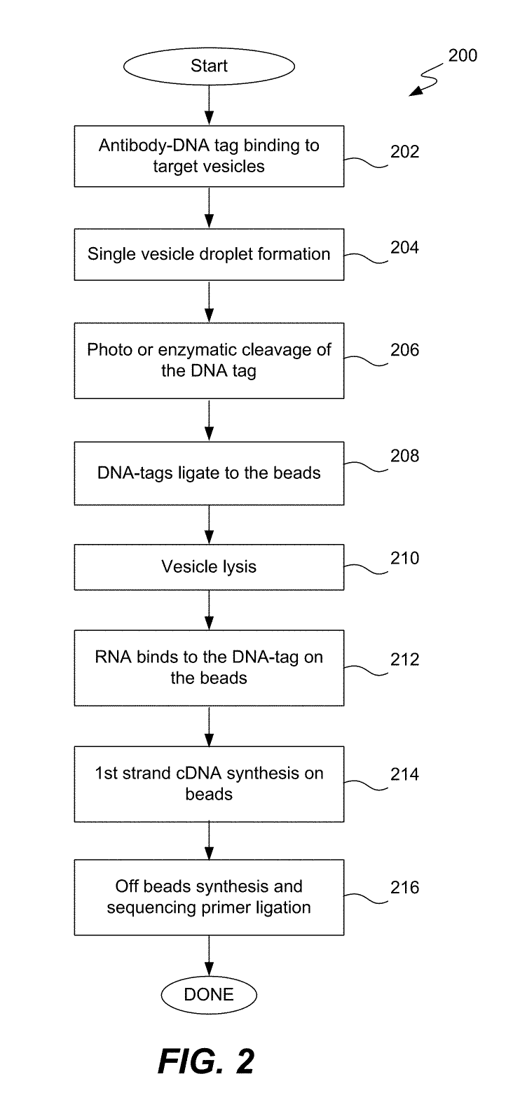

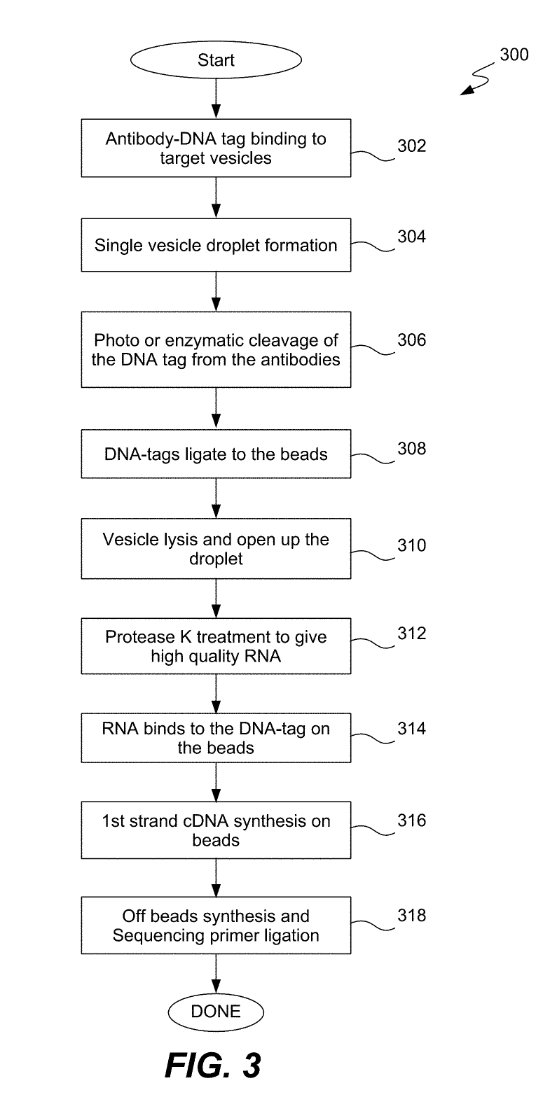

According to various embodiments of the present disclosure, FIG. 1 illustrates a flow process 100 of a first implementation for identification of EV subpopulations. FIG. 2 illustrates a flow process 200 of a second implementation for identification of exosome subpopulations, in accordance with one or more embodiments. FIG. 3 illustrates a flow process 300 of a third implementation for identification of EV subpopulations, in accordance with one or more embodiments.

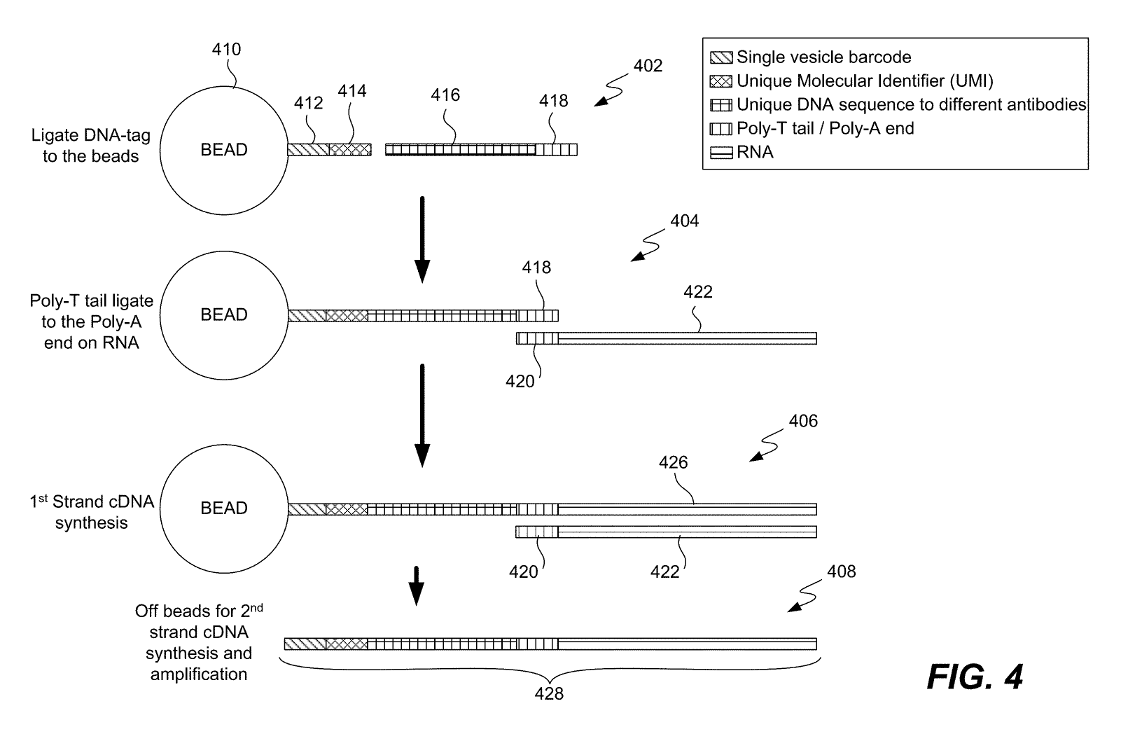

The first implementation may be referred to herein as “Version 1.” The second implementation may be referred to herein as “Version 2.” The third implementation may be referred to herein as “Version 3.” In some examples, Version 1 uses DNA tags that are unique to each vesicle; and thus each DNA tag will never be found in more than one droplet and can be used to identify oligonucleotides in a single droplet. In some examples, Versions 2 and 3 use DNA tags that are also unique to each vesicle, but RNA ligation via a poly-A tail is required for RNA capture inside the droplet; thus only RNA is measured that is vesicle derived, as the DNA tag confirms the presence of the EV surface protein (and thus the membrane), and the RNA ligation step confirms the presence of RNA inside the vesicle. In the following descriptions, process 100 of Version 1, process 200 of Version 2, and process 300 of Version 3 may include one or more similar or identical steps. FIGS. 1-3 illustrate flow charts for Versions 1-3, respectively. Thus, as used herein, Versions 1-3 will be synonymous with FIGS. 1-3, respectively. As described herein, Versions 1 and 2 will be described together with variations in the processes being noted. Additional steps of Version 3 will then be discussed. FIG. 4 illustrates the various configurations of a barcoded bead 400 with DNA-tags, in accordance with one or more embodiments. FIG. 4 will be described in conjunction with processes 100, 200, and 300.

EV Collection

In Versions 1 and 2, samples of EVs may first be prepared. First EVs may be harvested from cell culture, or any body fluid, e.g., blood. In the described examples, the EVs can be collected using polymer-based precipitation methods, size exclusion chromatography, ultrafiltration, ultracentrifugation, immunoaffinity method, or a combination of any of the named methods. The majority of contaminants (e.g., immunoglobin proteins in the serum or proteins from the cell culture media) from the fluid sample are removed during the exosome collection process. In order to further remove the exosome-free RNA from the fluid, the fluid from cell culture or any body fluid can be treated with RNAse to remove all the EV-free RNA. After treating with the RNAse, it is important to inactivate the RNAse in order for it not to interfere in the downstream processes of exosomal RNA isolation and detection. The RNAse may be inactivated by resuspending the isolated exosome in DEPC (Diethylpyrocarbonate)-containing PBS. Alternatively RNAse may be removed using an affinity purification column or gel filtration.

DNA-tagged Antibody Production

A selection of DNA-conjugated antibodies is then prepared to label the surface proteins on the exosomes using immunoaffinity (antibody-antigen binding). The process of creating the DNA-tagged antibodies consists of the following steps and includes techniques to confirm that each step was successful.

Antibodies against the desired EV membrane targets are selected. Generally commercially available antibodies may be selected. The specificity of these antibodies may be confirmed prior to any following steps. Specificity confirmation can be achieved in several ways, for example fluorescence microscopy can be used to image EVs or cells which are known to express the target antigen which are immobilized on a glass slide, stained with the selected antibodies and followed by fluorescently labeled secondary antibodies.

The desired oligonucleotide sequences may be manufactured and obtained from one of a wide array of suppliers. The DNA must be modified with functional chemical groups that can conjugate with the antibody. Often a photocleavable linker will be utilized so that the DNA can be easily released. Note that the photocleavable modification is commercially available on any DNA or RNA sequence. A photolabile functional group (a 10-atom linker) can be cleavable by UV light (300-350 nm) and the resulting oligonucleotide will have a 5′ phosphate group that is available for subsequent ligase reaction.

The oligonucleotide sequence is conjugated to the antibody. Multiple strategies exist for DNA-antibody conjugation including non-covalent strategies, such as coupling via biotin-streptavidin or covalent conjugation, e.g. thiol-maleimide chemistry. To confirm successful conjugation of DNA tags to the antibodies, an SDS-PAGE procedure can be performed to measure changes in molecular weight of the conjugates; a shift to higher molecular weight indicates successful conjugation.

Barcode Production

In the described embodiments, barcodes may be manufactured in a few different ways. Barcoded beads or hydrogels can be made by repeatedly pooling, splitting, and adding a single monomer to each fraction; after just 12 repeated cycles, over 16 million unique barcoded beads can be created in this way. As shown in FIG. 4, bead 410 includes a single vesicle barcode 412 with a unique molecular identifier 414. An alternate method is to make a barcode emulsion on a microfluidic chip by first taking a solution of single stranded DNA of random 10-mer sequences, mixing with PCR reagents and primers, and injecting through a droplet maker. The droplets are then thermocycled to amplify the single-molecule templates into a clonal population.

EV Surface Protein Labeling

The EVs may then be labeled and barcoded. At step 102, the EV surface proteins are labeled by binding anti-body-DNA tags to target proteins on vesicles. This step labels the surface proteins on single EVs using immunoaffinity (antibody-antigen binding). In some embodiments, the DNA tags may be configured to bind with specific proteins within the target vesicles. The purified EVs of interest are incubated with the cocktail of DNA-conjugated antibodies containing a photocleavable linker to allow DNA release after exposure to ultraviolet light. The antibodies will bind to the target proteins on the EV surface. The unbound DNA-conjugated antibodies can be removed again using polymer-based EV pulldown assay or size filtration/exclusion method. In various embodiments, the EV collection method here should be a general, unbiased method, aiming to remove the unbound DNA-conjugated antibodies and retain all the integral EVs.

Single-Vesicle Droplet Generation

At step 104, single-vesicle droplet is formed. In various embodiments, the DNA-antibody bound EVs, barcoded beads, and the reagents (i.e., ligase solution) are loaded in a commercially available droplet generator (i.e., BioRad system) or homemade microfluidic droplet generating device. Single EVs with single barcoded beads and the reagents are co-encapsulated into one droplet. It is noted that diluted EV samples are needed to avoid two or more EVs in the same droplet; this is a similar concept found in single cell RNA-seq to avoid two cells in the same droplet.

Enzymatic Cleavage of DNA-Tags

At step 106, photocleavage is used to cleave DNA-tags from antibodies in the droplet. To break the DNA-tags from the antibodies, the bonds between the DNA-tag and the antibody are photo-breakable linkers. DNA-tags are then released from the antibodies using light (e.g., >300 nm UV light which does not harm DNA or RNA). In some embodiments, enzymes can be used to cleave enzymatic linkages between DNA-tags from antibodies in the droplet instead of light.

DNA-Tag Ligation to Barcoded Beads

Switching briefly to FIG. 2, a unique step 208 is described here. Steps 202, 204, and 206 in FIG. 2 are analogous to steps 102, 104, and 106 in FIG. 1. At step 208, the DNA tags are ligated to the barcoded beads. This step does not occur in Version 1. This is because in version 1, the DNA-tag does not serve as linkage for the RNA to bind; the RNA binds to the beads directly. The released DNA-tags are then ligated to the barcode sequence on the beads by a ligase. For example, the ligase used in the present example may be T4 DNA ligase, which is responsible for binding DNA-tag (e.g., a double strand DNA) to DNA barcode sequence (e.g., a double strand DNA). It is noted that the ligase and its solution (e.g., ATP containing buffer) may be encapsulated with the EVs and barcoded beads from the moment of droplet creation. Alternatively, the DNA-tag sequence can be designed to have 5′ end which can specifically hybridize to the common sticky end sequence on the barcode sequence on the beads, middle region specific to the antibody, and the 3′ end to have poly T-tail for subsequent EV RNA binding. As illustrated in FIG. 4, at configuration 402, DNA antibody tag 416 that is unique to a specific antibody (and thus vesicle surface protein) with poly-T tail 418 is ligated to bead 410 via a single vesicle barcode 412 with a unique molecular identifier 414.

In-Droplet EV Lysis

At step 108, the EV in the droplet is lysed. In some embodiments, a detergent-based reagent may be used to lyse the vesicles. After photo (or enzymatic) cleavage of the DNA-tags from antibodies at step 106, or after ligating the DNA-tag to the barcoded beads at step 208 a new line of buffer containing EV lysis reagents and reverse transcription (RT) reagents is injected to each droplet encapsulated both single EV and single DNA-tag bound barcoded bead. The RT-EV lysis reagent mix serves as a single buffer containing reagents to lyse the EVs (such as detergent) and reagents to perform reverse transcription (such as reverse transcriptase for transcribing RNA into cDNA). RNAaseOUT recombinant ribonuclease inhibitor (e.g. a noncompetitive inhibitor of ribonucleases such as RNAase A) or other RNAse removal reagent can also be included in the buffer for reducing any chances of RNA degradation by RNAse.

In-Droplet EV RNA Hybridizing to the DNA-Tagged Beads

At step 110, the EV RNA is hybridized to the DNA-tagged beads. In some embodiments, as the EVs are lysed inside each droplet, and the released EV RNA will hybridize on the poly-T tail of the DNA-tag. In eukaryotes (e.g., human cells), polyadenylation is part of the process that produces mature messenger RNA (mRNA) for translation. The mRNA can be packaged into the EVs in the human cells. Thus, a DNA sequence with poly-T-tail can be used to effectively capture mRNA in EVs. Additionally, pre-miRNAs are transcribed by RNA polymerase II and comprise of a 5′-cap and poly-A tail as well. Long non coding RNAs (lincRNAs) also predominantly contain poly A tails; thus any RNA species with a poly A tail could be hybridized in step 110. As illustrated in FIG. 4, at configuration 404, the poly-T tail 418 is ligated to the poly-A end 420 of RNA 422.

In some embodiments, the DNA barcode sequence on the beads can be designed to have a poly-T end for hybridizing to the EV RNA (with poly-A tail). In such embodiments, the DNA-tag conjugated with antibodies can also be designed to have the poly-A-tail to hybridize (instead of ligate) to the poly-T sequence on the DNA barcode.

Additionally, in some embodiments, to ensure capture of the mature RNAs, which do not have poly-A-tails, we can perform polyadenylation process to the RNAs before the hybridization process. For example, for polyadenylating miRNAs with no poly A tail, an miRNA cDNA synthesis kit including poly(A) polymerase which catalyzes the template independent addition of adenosine residue onto the 3-end of polyribonucleotides is used. After using this kit, the miRNA will be poly-A-tailed.

In-Droplet cDNA Synthesis

At step 112, cDNA is synthesized in the droplet. After binding of EV RNA on the barcoded beads (either through the DNA-tag or not, as in Version 1), the existing reverse transcriptase in the droplet will start the first strand cDNA synthesis at designated temperature. As illustrated in FIG. 4, at configuration 406, first strand cDNA 426 is synthesized from RNA 422. As illustrated in FIG. 1, Version 1 may include an additional step 114 in which second strand cDNA is synthesized on the barcoded bead. As Illustrated in FIG. 2, Version 2 may include off-bead synthesis and sequencing primer ligation step 216 after step 214. In some examples of Versions 1 and 2, all the aforementioned steps, from single vesicle droplet formation 104 or 204, to first strand cDNA synthesis 112 or 214, is completed inside each droplet. In some examples of Version 1, cDNA synthesis is not conducted if vesicle-derived DNA instead of RNA is captured.

Droplet Breakage and Sample Pooling

After first strand cDNA synthesis, the beads have a DNA sequence including the barcode sequence (originally on the beads), the DNA-tag (released from the antibody), and the cDNA sequence (transcribed from the EV RNA). The barcode sequence comprises information regarding the EV-of-origin because it identifies a surface protein that was present on that single EV; EVs from different cells and different disease states can contain different surface proteins. The DNA-tag comprises information regarding the specific antibody and the UMI (unique molecular identifier) for identifying copy numbers. Since all this information is encoded within each bead, the droplet can now be broken and the samples can be pooled for bulk analysis while maintaining identification of which barcodes (and thus DNA tags and cDNA sequences) originated from which single EV. Since the immunomagnetic beads are used here, a magnet may be used to pull down the beads. The DNA sequences on the beads will then be purified and released from the beads.

In some embodiments, Version 1 can be used to capture both or either RNA or DNA. Because Version 1 does not require the antibody DNA-tag to serve as an anchoring point (poly-T) to the RNA (poly-A), and thus any DNA or RNA could adhere to the beads. DNA or RNA measurement would then be decided by the choice of downstream library preparation kits (e.g. a kit that purifies and prepares DNA or a kit that purifies and prepares DNA.)

Creation of Library and Sequencing and Readout

In various embodiments, commercially available kits and protocols may be used to create a library and sequencing and readouts.

Version 3, as illustrated in FIG. 3, shares one or more steps with Versions 1 and 2, as depicted in FIGS. 1 and 2, respectively. In the described embodiments, Version 3 includes the same preparation steps for the EV samples, including EV collection, RNAse treatment of EVs, DNA-tagged antibody production, and barcode production. For example, steps 302-306 are analogous to steps 102-106 of FIG. 1 or 202-206 of FIG. 2.

FIG. 3 may include a step 302 for EV surface protein labeling. Within a well, the immunoprobe targets EVs with (>20) different antibodies labelled with unique DNA-tags. The DNA tags are configured to contain both unique sequences corresponding individual antibodies and specific sequences needed for following RNA ligation to the beads. It is important for this dual measurement to eliminate the reads from EV proteins derived from EVs encapsulated with no oligonucleotide, vesicle-free proteins, or vesicle-free RNAs. This provides an inherent quality control function. The well may then be washed to remove any unbound antibodies. In some embodiments, this step can be performed by conventional bead or polymer-based pulldown.

FIG. 3 may also include step 304 for forming a single-vesicle droplet. EVs are encapsulated with droplets containing barcoded magnetic beads and enzymes if enzymatic cleavage is performed. Version 3 may also include step 306 for enzymatic or photocleavage of DNA-tags. Enzymes are used to cleave DNA-tags from antibodies in the droplet.

At step 308, DNA ligase ligates DNA-tags onto the barcoded magnetic beads in the droplet, such as in step 208. Individual droplets may further be settled in individual microwells of the chip at 308. The single magnetic bead and single EV containing droplets can be directed into individual microwells on a microfluidic chip using an acoustofluidic platform. When surface acoustic waves are applied to the device, each droplet will be pushed into the microwells inside the microchannel. The usage of the magnetic beads is configured for enhanced settling efficiency and improved handling. The reason to capture the beads in microwells or otherwise immobilize them on a chip surface is to be able to remove them from the droplets and enable them to be more easily accessed the following steps described below. Alternatively, an electrowetting process can also be applied to direct individual droplets to move on the microfluidic chip and fall into the microwells.

The microwells are designed to fit single droplet. The microfluidic chip with the microwells can be fabricated using high density polyacrylamide gel against Si-mold. The high density polyacrylamide gel fabricated microwell is designed to prevent EV RNA from single EV to diffuse laterally and prevent contamination between neighboring EVs. A magnet will be applied at the bottom of the microfluidic chip throughout the process to keep magnetic beads at the bottom of each microwell. In some examples of Version 3, only steps 304, 306, and 308 need to be completed directly inside each droplet; subsequent steps can be completed off-droplet.

At step 310 in Version 3, a detergent-based reagent may be used to lyse the vesicles and break the droplets on the chip. After settling each droplet into each microwell, lysis buffer solution will be applied to break the droplets. The same lysis buffer will also serve as the lysis reagent to lyse the EVs and extract EV RNA in the microwells. As described, a magnetic stand may be used to keep the beads at the bottom of the chip. At step 312, the beads are treated with Proteinase K to unbound RNAs from any proteins, which allows only high quality RNAs to remain. Proteinase K can be added into the lysis buffer to ensure any binding protein on the EV RNA and ensure maximum EV RNA extraction. Since all the samples are trapped in the microfluidic device now with a magnet, the reagents can be easily applied by pouring or pipetting the buffer directly to the chip. Additionally, the wash step can also be easily performed by pouring or pipetting the wash solution.

At step 314, single EV RNAs are bound to the DNA-tags on the barcoded magnetic beads. This step may be similar to step 110 of Version 1 and 212 of Version 2, which occurs within a droplet. The extracted EV RNA can now hybridize to the DNA-tagged beads via the poly A-tail on the RNA and the poly-T-tail end on the DNA-tag.

1st strand cDNA synthesis may then be performed on beads, or on the chip, at step 316. As illustrated in FIG. 4, at configuration 406, first strand cDNA 426, with complementary Poly-A end 424, is synthesized from RNA 422. After EV RNA binding to the DNA-tagged magnetic beads, the first strand cDNA synthesis buffer can be applied and the reverse transcription and cDNA synthesis will start. In some embodiments, the first strand cDNA synthesis on the beads, or the chip, may then be purified. This may be similar to step 112 and 214 of Versions 1 and 2, which occurs within a droplet. All samples are then pooled by collecting all the beads together for easier downstream handling since all the samples are individually barcoded. As Illustrated in FIG. 3, Version 3 may include off-bead synthesis and sequencing primer ligation step 318 after step 316.

In various embodiments, off bead synthesis and sequencing primer ligation may be implemented. In some embodiments, second strand cDNA synthesis may also be performed, such as in step 114, and the second strand cDNA synthesis is purified. The cDNA may be tagmented to create sequencing libraries, and amplified to amplify both protein and RNA signals. The sequencing libraries are then purified. Next-generation sequencing may then begin. As illustrated in FIG. 4, configuration 408 shows an off bead 428 for second strand cDNA synthesis and amplification.

In various embodiments, commercially available kits and protocols may be used to create a library and sequencing and readouts, as in Versions 1 and 2.

In Version 1, the protein and RNA (or DNA) “tags” that refer back to a unique vesicle are not “coordinated”. As such, the tags may refer back to the same single vesicle, but they themselves may not be the same between the protein on the EV surface and the internal RNA (or DNA). In Versions 2 and 3, the surface protein and RNA tags are coordinated so that a “readout” is only provided if an EV contains both protein and RNA (or DNA, depending on the experiment at hand). This is especially important for EVs because many EVs are “blanks,” which contain no internal cargo. Thus, a mechanism where no downstream next-generation sequencing (NGS) is done if an EV does not have both: 1.) one or a combination of surface proteins and 2.) the presence of a nucleic acid. This results in more efficient NGS and cost savings, as NGS is not run when there is simply no nucleic acids present. All of the protein and RNA tagging steps then must occur inside the single droplet that contains the single EV.

In various embodiments, specialized hardware may be used to improve or enable this workflow. Such hardware may include: microfluidic immunomagnetic EV isolation and enrichment technology for optimized yield and purity, DNA barcoded bead and antibody reagents, custom instrumentation for droplet processing, and microwell-based chip design for droplet isolation and downstream handling.

In some embodiments, linking of antibody-DNA tag to RNA to reduce extraneous counts and enhance EV signal may enhance the workflow mainly by reducing NGS read counts, which have significant time and cost associated with them.

The described systems and methods provide various benefits for identifying EV subpopulations and payloads for potential targeted therapeutics. First, simultaneously measuring EV surface proteins and RNA de novo discovery of RNA payloads associated with particular subgroups of EVs defined by their surface markers. This unique measurement capability applied to clinical liquid biopsy samples and merged with big data science will enable the discovery of new targeted therapeutics.

Secondly, the use of DNA tags provides several additional advantages that are particularly advantageous for EVs compared to single cells: the ability to amplify and thereby detect low abundance tags, to correct read counts using UMIs, and essentially provide unlimited multiplexing potential. For example, the Helios CyTOF system (Fluidigm) can detect down to 350 antibodies/cell, and the FACSAria III Cell Sorter (BD) can detect 85 FITC molecules/particle, according to the manufacturers' specification. With DNA antibody tags one can theoretically detect a single antibody per cell or vesicle because the DNA tags are amplified from single-molecule templates.

Furthermore, the unique design of the DNA-tag allows dual EV surface protein and RNA measurement, eliminating the contamination of other protein aggregates or circulating vesicle-free RNAs from the clinical samples. This purification step maximizes the throughput by reducing the sequencing reads used for contaminant sequences. By coupling surface protein measurement with internal nucleic acid measurement for single EVs, sequencing EVs that contain no internal nucleic acids is avoided. A large percentage of EVs, in particular from a human blood sample or cell culture sample, may not contain internal nucleic acids (referred to herein as “blank” EVs). But most technologies cannot prevent these “blank” EVs from being sequenced because it would become quite cost prohibitive. This is not an issue in single-cell RNA-seq in the described systems and methods, where cells by definition always contain some type of nucleic acid inside.

Additionally, removing the reactions or handling steps from the droplets will facilitate better cleanup of RNA samples (i.e., proteinase K treatment) before the cDNA synthesis step. Proteinase K cannot be included in the droplets as it will remove all the necessary enzymes needed for DNA or RNA ligation to the beads if enzymatic cleave is used

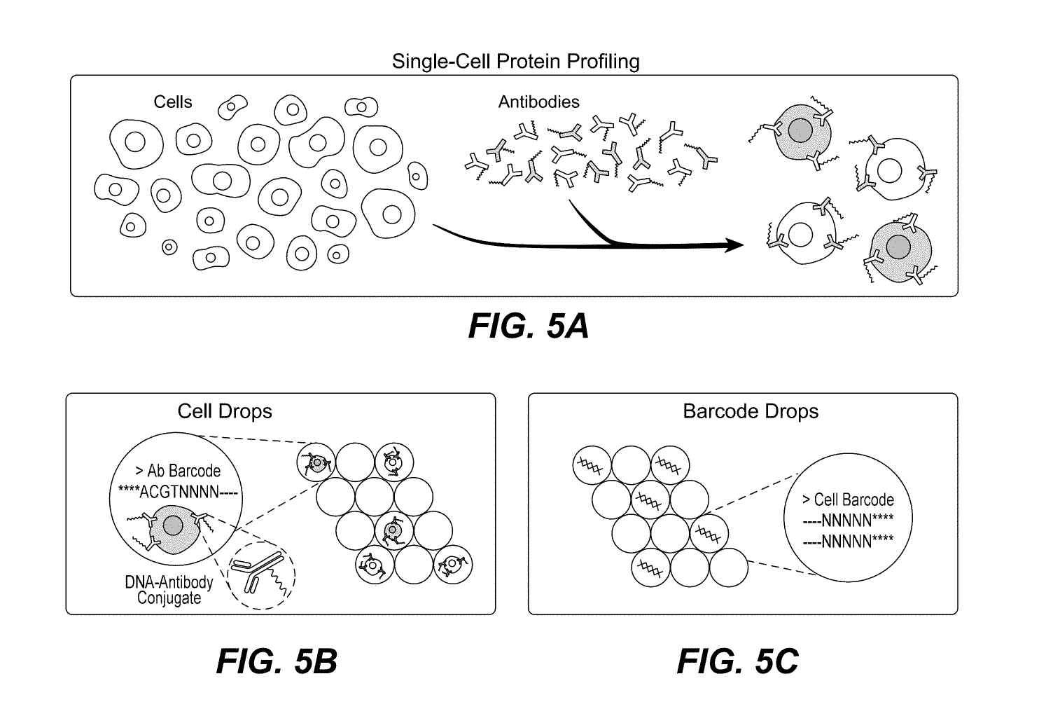

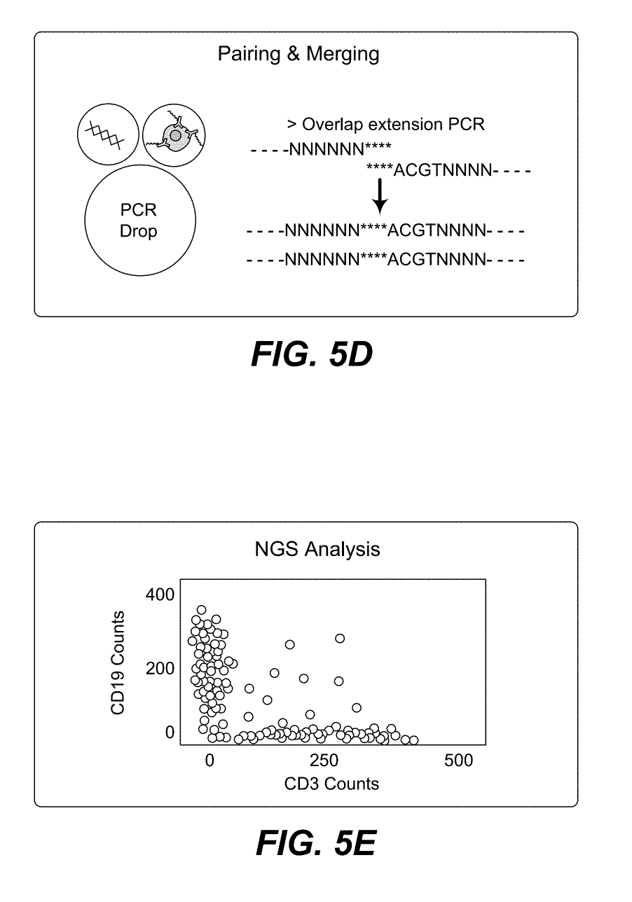

FIG. 5A-5E illustrate a method for single-cell protein profiling, in accordance with one or more embodiments. FIG. 5A illustrates DNA-antibody conjugates are introduced to target cells to cause binding. FIG. 5B illustrates cell drops are formed which include the DNA-antibody conjugate bound to a target cell. FIG. 5C illustrates barcode drops are also formed. FIG. 5D illustrates the cell drops and barcode drops are paired and merged. FIG. 5E illustrates an NGS analysis is performed.

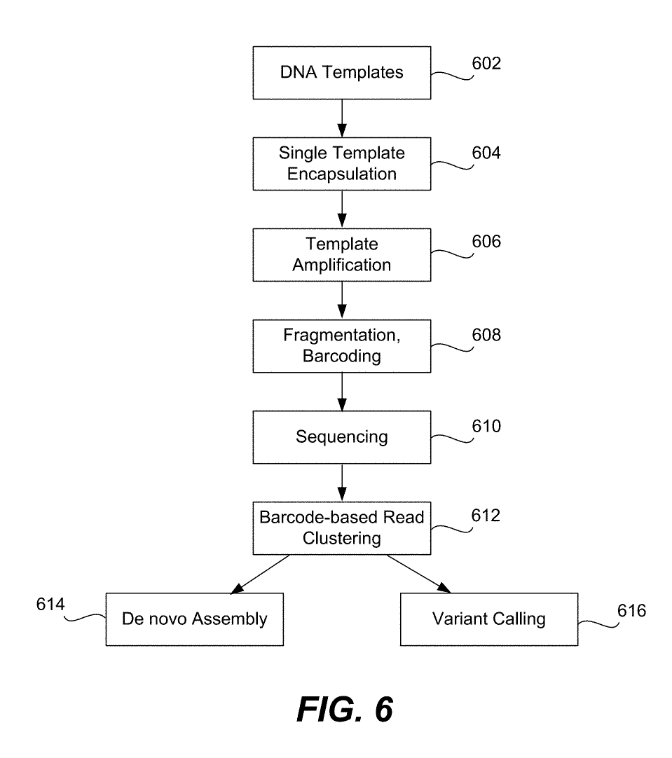

FIG. 6 illustrates a method 600 for single-molecule droplet barcoding (SMDB), in accordance with one or more embodiments. DNA templates are generated (602) and then encapsulated (604) into droplets such that most droplets contain zero or one template. Templates are clonally amplified (606) to produce multiple copies in each droplet. Templates are fragmented (608) inside drops, and barcodes are attached (608) to fragments such that each droplet gets a unique barcode sequence. All fragments are sequenced (610) in parallel and resulting reads are clustered (612) based on barcode. Clustered reads are used to reconstruct (614) the sequence or accurately detect (616) SNPs for the template encapsulated in each droplet.

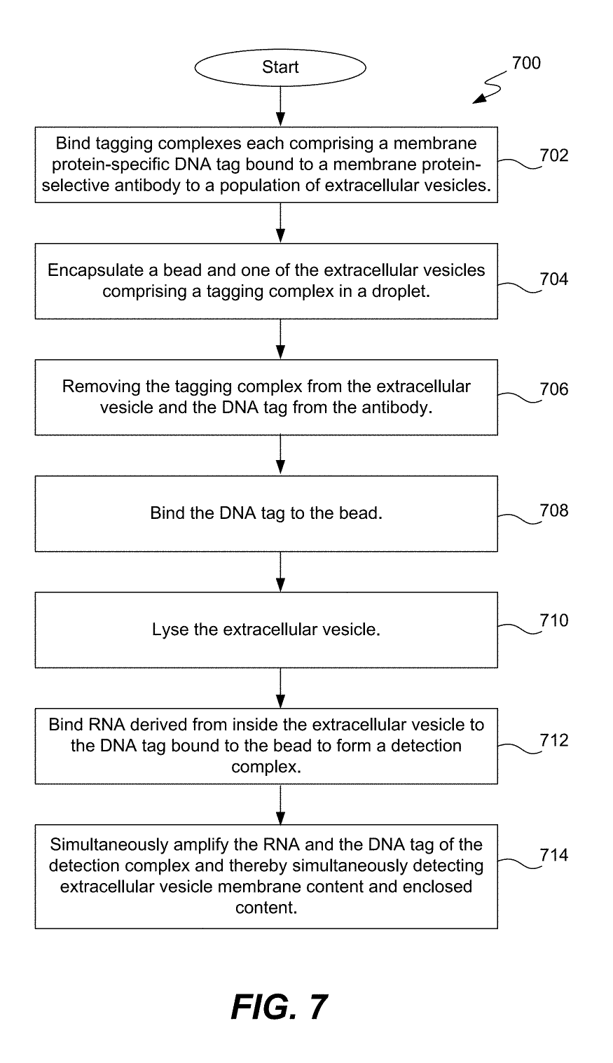

FIG. 7 illustrates a method 700 for simultaneously detecting extracellular vesicle membrane and enclosed content, in accordance with one or more embodiments. At 702, tagging complexes each comprising a membrane protein-specific DNA tag bound to a membrane protein-selective antibody are bound to a population of extracellular vesicles. At 704, a bead and one of the extracellular vesicles comprising a tagging complex is encapsulated in a droplet. At 706, the tagging complex from the extracellular vesicle and the DNA tag are removed from the antibody. At 708, the DNA tag is bound to the bead. At 710, the extracellular vesicle is lysed. At 712, RNA derived from inside the extracellular vesicle is bound to the DNA tag bound to the bead to form a detection complex. At 714, the RNA and the DNA tag of the detection complex are simultaneously amplified and thereby extracellular vesicle membrane content and enclosed content are simultaneously detected.

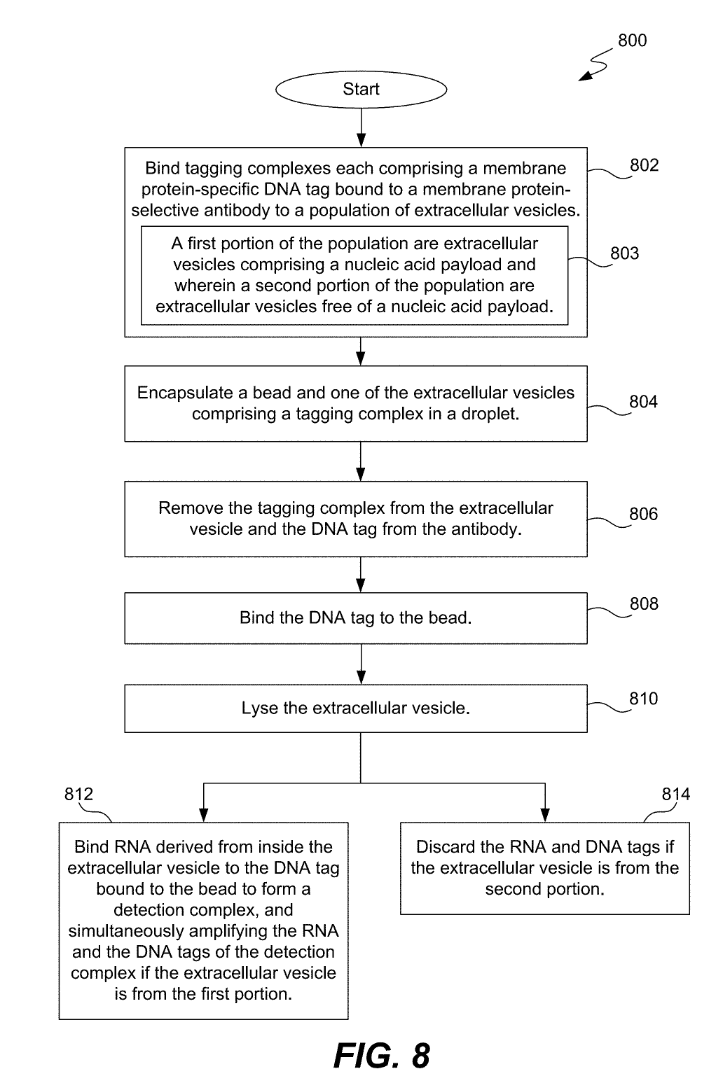

FIG. 8 illustrates another method 800 for detecting extracellular vesicle content, in accordance with one or more embodiments. At 802, tagging complexes each comprising a membrane protein-specific DNA tag bound to a membrane protein-selective antibody are bound to a population of extracellular vesicles. In some embodiments, a first portion of the population are extracellular vesicles comprising a nucleic acid payload and a second portion of the population are extracellular vesicles free of a nucleic acid payload (803). At 804, a bead and one of the extracellular vesicles comprising a tagging complex are encapsulated in a droplet. At 806, the tagging complex from the extracellular vesicle and the DNA tag are removed from the antibody. At 808, the DNA tag is bound to the bead. At 810, the extracellular vesicle is lysed. After step 810, method 800 can proceed to either step 812, if the extracellular vesicle is from the first portion, or 814, if the extracellular vesicle is from the second portion. At 812, RNA derived from inside the extracellular vesicle is bound to the DNA tag bound to the bead to form a detection complex. In addition, the RNA and the DNA tags of the detection complex are simultaneously amplified. At 814, the RNA and DNA tags discarded.

Although many of the components and processes are described above in the singular for convenience, it will be appreciated by one of skill in the art that multiple components and repeated processes can also be used to practice the techniques of the present disclosure.

While the present disclosure has been particularly shown and described with reference to specific embodiments thereof, it will be understood by those skilled in the art that changes in the form and details of the disclosed embodiments may be made without departing from the spirit or scope of the disclosure. It is therefore intended that the disclosure be interpreted to include all variations and equivalents that fall within the true spirit and scope of the present disclosure.

What is claimed is:

1. A simultaneous extracellular vesicle membrane and enclosed content detecting composition comprising:
a. a bead comprising a selective binding agent;
b. an extracellular vesicle membrane protein-specific DNA tag bound to the selective binding agent and comprising a poly-T end;
c. an extracellular vesicle-derived RNA comprising a poly-A end bound to the membrane protein-specific DNA tag poly-T end.

2. The composition according to claim 1, further comprising cDNA bound to the RNA.

3. The composition according to claim 1, wherein the selective binding agent comprises a barcode and a molecular identifier.

4. The composition according to claim 1, wherein the selective binding agent selectively binds the membrane protein identifier DNA present in a droplet.

5. The composition according to claim 1, wherein the bead is a magnetic bead.

6. The composition according to claim 1, wherein the bead is a conductive bead.

7. A method of simultaneously detecting extracellular vesicle membrane and enclosed content, the method comprising:
a. binding tagging complexes each comprising a membrane protein-specific DNA tag bound to a membrane protein-selective antibody to a population of extracellular vesicles;
b. encapsulating a bead and one of the extracellular vesicles comprising a tagging complex in a droplet;
c. removing the tagging complex from the extracellular vesicle and the DNA tag from the antibody;
d. binding the DNA tag to the bead;
e. lysing the extracellular vesicle;
f. binding RNA derived from inside the extracellular vesicle to the DNA tag bound to the bead to form a detection complex;
g. simultaneously amplifying the RNA and the DNA tag of the detection complex and thereby simultaneously detecting extracellular vesicle membrane content and enclosed content.

8. The method according to claim 7, further comprising isolating the population of extracellular vesicles.

9. The method according to claim 7, wherein a portion of extracellular vesicles of the population of extracellular vesicles are free of encapsulated RNA.

10. The method according to claim 7, wherein the amplification is only performed on samples in droplets comprising an RNA derived from inside the extracellular vesicle and a DNA tag.

11. The method according to claim 7, wherein the amplification is preferentially performed on samples in droplets comprising an RNA derived from inside the extracellular vesicle and a DNA tag.

12. The method according to claim 7, wherein the DNA tag comprises a poly-T end and the RNA comprises a poly-A end, and wherein binding the RNA to the DNA tag comprises binding the poly-T end of the DNA tag with the poly-A end of the RNA.

13. The method according to claim 7, further comprising isolating a droplet comprising a bead and one of the extracellular vesicles comprising a tagging complex in a well of a droplet isolating device.

14. The method according to claim 13, wherein the bead is a magnetic bead and the droplet isolating device comprises a magnet that positions the beads inside the well.

15. The method according to claim 13, further comprising purifying RNA derived from inside the extracellular vesicle after the extracellular vesicle is lysed.

16. The method according to claim 15, wherein lysing the extracellular vesicle produces a lysed solution and wherein purifying the RNA comprises treating the lysed solution with proteinase K.

17. The method according to claim 7, further comprising cleaving the detection complex from the bead before simultaneously amplifying the RNA and the DNA tag.

18. The method according to claim 7, wherein the extracellular vesicles are exosomes.

19. The method according to claim 7, wherein the extracellular vesicles are nanovesicles.

20. A method of detecting extracellular vesicle content, the method comprising:
a. binding tagging complexes each comprising a membrane protein-specific DNA tag bound to a membrane protein-selective antibody to a population of extracellular vesicles, wherein a first portion of the population are extracellular vesicles comprising a nucleic acid payload and wherein a second portion of the population are extracellular vesicles free of a nucleic acid payload;
b. encapsulating a bead and one of the extracellular vesicles comprising a tagging complex in a droplet;
c. removing the tagging complex from the extracellular vesicle and the DNA tag from the antibody;
d. binding the DNA tag to the bead;
e. lysing the extracellular vesicle; and
f. binding RNA derived from inside the extracellular vesicle to the DNA tag bound to the bead to form a detection complex, and simultaneously amplifying the RNA and the DNA tags of the detection complex if the extracellular vesicle is from the first portion; or
g. discarding the RNA and DNA tags if the extracellular vesicle is from the second portion.
